(19) United States
(12) Patent Application Publication
(10) Pub. No.: US 2008/0234753 A1
(43) Pub. Date: Sep. 25, 2008

(54) Spinal Stabilization Systems

(75) Inventor: Hai H. Trieu, Cordova, TN (US)

Correspondence Address: COATS & BENNETT/MEDTRONIC, 1400 CRESCENT GREEN, SUITE 300, CARY, NC, 27518, US

(73) Assignee: Warsaw Orthopedic, Inc., Warsaw, IN (US)

(21) Appl. No.: 11/687,732

(22) Filed: Mar. 19, 2007

(51) Int. Cl. A61B 17/70; A61B 17/58

(52) U.S. Cl. 606/297; 606/283; 606/286

(57) ABSTRACT

A stabilizing system for stabilizing a patient's spinal column comprises a flexible implant configured for attachment to a vertebral member, an anchor plate for attaching the flexible implant to the vertebral member, and at least one fastener to secure the anchor plate to the vertebral member. The anchoring plate has a bottom surface with one or more projections configured to penetrate the top surface of the flexible implant when a clamping force is applied.

### BACKGROUND

The present application is directed to devices and methods for spinal stabilization and fixation.

The spine is divided into four regions comprising the cervical, thoracic, lumbar, and sacrococcygeal regions. The cervical region includes the top seven vertebral members identified as C1-C7. The thoracic region includes the next twelve vertebral members identified as T1-T12. The lumbar region includes five vertebral members L1-L5. The sacrococcygeal region includes nine fused vertebral members that form the sacrum and the coccyx. The vertebral members of the spine are aligned in a curved configuration that includes a cervical curve, thoracic curve, and lumbosacral curve. Intervertebral discs are positioned between the vertebral members and permit flexion, extension, lateral bending, and rotation.

Spinal implants are often used in the surgical treatment of spinal disorders such as degenerative disc disease, disc herniations, curvature abnormalities, and trauma. Many different types of treatments are used. In some cases, spinal fusion is indicated to inhibit relative motion between vertebral members. In other cases, dynamic implants are used to preserve motion between vertebral bodies. In yet other cases, relatively static implants that exhibit some degree of flexibility may be attached to the vertebral members.

The spinal implants may include a flexible implant, such as a tether or a plate, that extends between two or more vertebral members. The flexible implant is positioned against the vertebral members and held in position by one or more anchors. The anchors should extend through the implants in a manner to prevent damage that could cause the implant to fail.

### SUMMARY

The present application is directed to devices and methods for spinal stabilization and fixation. One exemplary embodiment of the stabilization system comprises a flexible implant configured for attachment to a vertebral member, an anchor plate for attaching the flexible implant to the vertebral member, and at least one fastener to secure the anchor plate to the vertebral member. The anchoring plate has a bottom surface with one or more projections configured to penetrate the top surface of the flexible implant when a clamping force is applied. The anchor plate distributes the loads more evenly over the flexible implant to minimize or reduce damage to and increase the service life of the flexible implant.

### BRIEF DESCRIPTION OF THE DRAWINGS

FIG. 1 is a side view of a spinal column.

FIG. 2 is an anterior view of an exemplary stabilization system.

FIG. 3 is a side view of an exemplary stabilization system.

FIGS. 4A-4C are anterior views of additional exemplary stabilization system.

FIGS. 5A and 5B illustrate exemplary flexible implants for a stabilization system.

FIGS. 6A-6D are sectional views of exemplary anchoring plates for use in a stabilization system.

FIGS. 7A-7D are sectional views of exemplary anchoring plates for a stabilization system.

FIGS. 8A-8D are elevation views of exemplary bone anchors for a stabilization system.

FIG. 9 is a top view of an exemplary anchor plate for a stabilization system.

FIG. 10 is a section view of an exemplary anchor plate for a stabilization system.

FIG. 11 is a section view of an exemplary anchor plate for a stabilization system with an exemplary locking device installed.

FIG. 12 is a section view of an exemplary anchor plate for a stabilization system.

FIG. 13 is a section view of an exemplary anchor plate for a stabilization system with an exemplary locking device installed.

### DETAILED DESCRIPTION

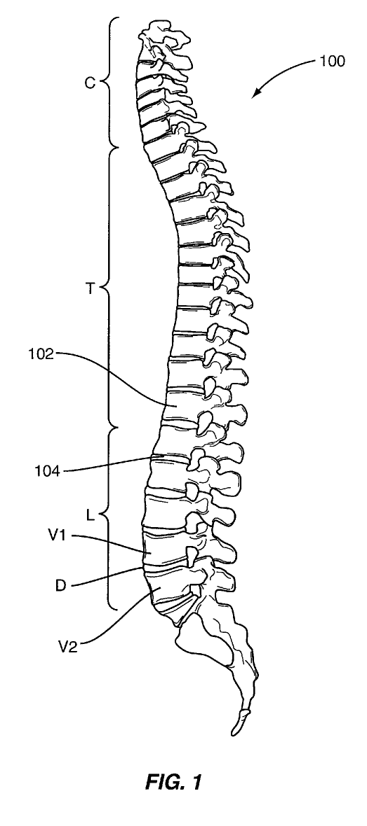

The present application relates to stabilizing systems for stabilizing vertebral members and to methods of applying the stabilizing system to a patient's spinal column 100. Referring first to FIG. 1, a side view of a spinal column is shown. The letters C, T and L refer to the cervical, thoracic and lumbar regions of the spine respectively. The spinal regions are made up of a series of vertebral members 102 separated by intervertebral discs 104.

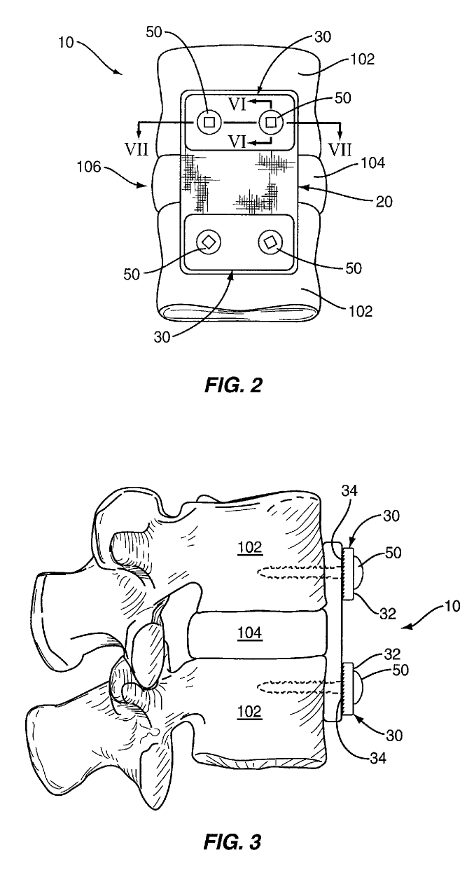

FIGS. 2 and 3 illustrate a vertebral joint 106 with an exemplary stabilization system 10 applied. FIG. 2 shows an anterior view of a vertebral joint 106 and FIG. 3 shows a side view of the vertebral joint 106. The vertebral joint 106 comprises two vertebral members 102 and an intervertebral disc 104. Although FIGS. 2 and 3 illustrate the stabilizing system spanning two vertebral members 102, other systems may span more than two vertebral members 102. The stabilizing system 10 may be applied to vertebral members 102 in the cervical, thoracic, and lumbar regions of the spinal column 100. The vertebral stabilizing system 10 may restore the functional support of a resected or damaged ligament by providing support against tension, torsion and shear forces on the joint 106.

The vertebral stabilizing system 10 extends between two or more vertebral members 102 and across the associated intervertebral discs 104. In some patients, the intervertebral discs 104 may have been fused or resected and replaced with a motion preserving device. As shown in FIGS. 2 and 3, the vertebral stabilizing system 10 comprises a flexible implant 20 such as a vertebral plate or tether, one or more anchor plates 30 to distribute clamping forces to the flexible implant 20, and one or more bone anchors 50 to secure each anchor plate 30 to a vertebral member 102.

In the embodiment shown in FIGS. 2 and 3, the stabilization system comprises two anchor plates 30 and four bone anchors 50. The bone anchors 50, which may for example comprise bone screws, penetrate into the vertebral member 102 and apply a clamping force to the anchor plate 30. The anchor plate 30 in turn distributes the clamping force over the surface of the flexible implant 20 and clamps the flexible implant 20 against the vertebral member 102. The anchor plate 30 may, in some embodiments, deform when the clamping force is applied to conform to the shape of the vertebral member 102.

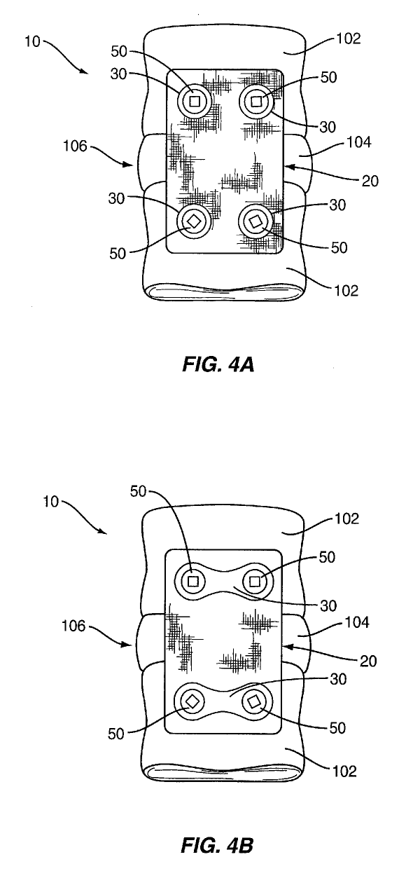

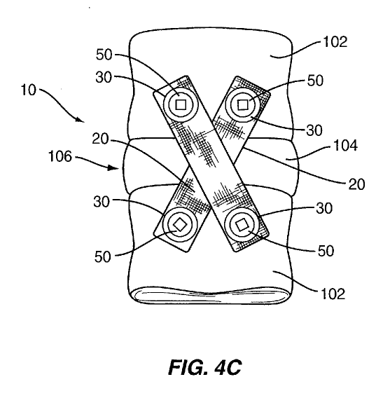

FIGS. 4A-4C show alternate embodiments of the stabilizing system 10. In the embodiment shown in FIG. 4A, the implant 20 is secured by four anchor plates 30 and four bone anchors 50. In this embodiment, there is a separate anchor plate 30 for each bone anchor 50. The anchor plates 30 are shaped like circular washers. Those skilled in the art will appreciate, however, that the anchor plates may be of any suitable form, such as square, triangular, hexagonal, octagonal, oval, elliptical, etc. The bone anchors 50 are located adjacent the four corners of the bone plate 22.

In the embodiment shown in FIG. 4B, opposing ends of the implant 20 are secured to respective vertebral members 102 by an hourglass-shaped anchor plate 30 and two bone anchors 50. The upper two bone anchors 50 penetrate the upper vertebral member 102, while the lower bone anchors 50 penetrate into the lower vertebral member 102. The narrow section of the hourglass-shaped anchor plate 30 allows the anchor plate 30 to more easily deform when the bone anchors 50 are tightened down on the top surface of the anchor plate 30.

In the embodiment shown in FIG. 4C, two flexible implants 20 in the form of a tether stabilize the vertebral joint 106. Opposing ends of each flexible implant 20 are secured to respective vertebral members 102. A single anchor plate 30 and bone anchor 50 is disposed at each end of each tether.

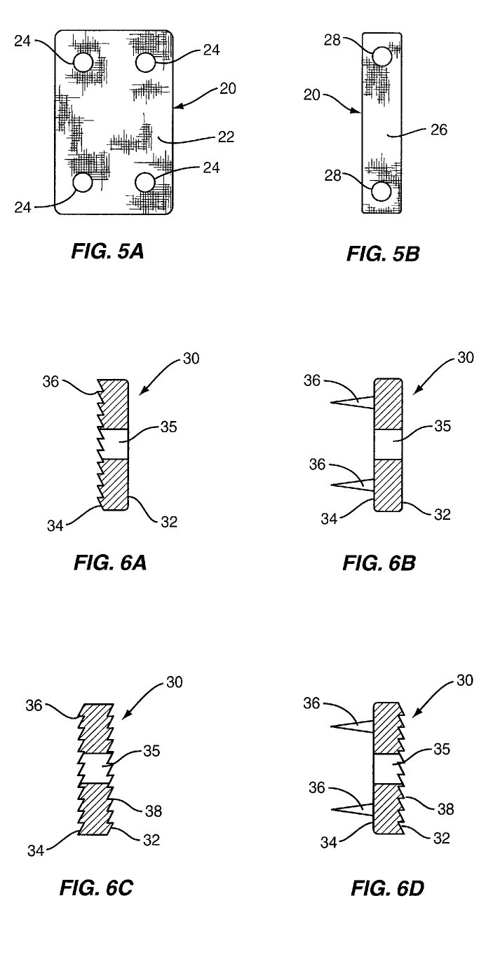

FIGS. 5A and 5B illustrate exemplary flexible implants 20. The flexible implant 20 in FIG. 5A comprises a generally rectangular bone plate 22 including four holes 24 through which the bone anchors 50 pass. The flexible implant 20 in FIG. 5B comprises a tether 26 including a hole 28 at each end thereof through which the bone anchor 50 passes. The flexible implant 20 is made of a flexible, biocompatible material, which may be elastic, inelastic, or semi-elastic.

Exemplary elastic materials for a flexible implant 20 include polyurethane, silicone, silicone-polyurethane, polyolefin rubbers, hydrogels, and the like. Other suitable elastic materials may include NITINOL or other superelastic alloys. Further, combinations of superelastic alloys and non-metal elastic materials may be suitable to form elastic strands. The elastic materials may be resorbable, semi-resorbable, or non-resorbable.

Exemplary inelastic materials for a flexible implant 20 include, for example, polymers, such as polyetheretherketone (PEEK), polyethylene terephthalate (PET), polyester, polyetherketoneketone (PEKK), polylactic acid materials (PLA and PLDLA), polyaryletherketone (PAEK), carbon-reinforced PEEK, polysulfone, polyetherimide, polyimide, ultra-high molecular weight polyethylene (UHMWPE), and/or cross-linked UHMWPE, among others. Metals or ceramics can also be used, such as cobalt-chromium alloys, titanium alloys, nickel titanium alloys, NITINOL, memory wire and/or stainless steel alloys, calcium phosphate, alumina, and/or pyrolytic carbon.

U.S. patent application Ser. No. 11/612,634 filed on Dec. 19, 2006 describes another flexible implant 20 that is suitable for use in the stabilization system described herein. This application is incorporated herein in its entirety by reference.

The anchor plate 30 may comprises a plate or disc of virtually any shape and made of a biocompatible material with sufficient rigidity to distribute clamping forces generated by the bone anchors 50 over the surface of the flexible implant 20. The anchor plate 30 includes a top surface 32 and a bottom surface 34. The bottom surface 34 of the anchor plate includes one or more penetrating members 36 for penetrating at least the top surface of the flexible implant 20. In some embodiments, the penetrating members 36 may pierce all the way through the anchor plate 30 and into the vertebral member 102 to supplement the clamping forces generated by the bone anchors 50. The penetrating members 36 may comprise teeth serrations, ridges, spikes, or similar features configured to pierce the surface of the flexible implant 20. The top surface 32 of the anchor plate 30 may be smooth, or may be textured to enhance the contact between the anchor plate and the bone anchor 50. Further, positive gripping features may be formed on the top surface of the anchor plate to prevent the bone anchors 50 from backing off as will be hereinafter described.

FIGS. 6A-6D are section views of exemplary anchor plates 30 taken through line VI-VI of FIG. 2 illustrating various penetrating members 36 for piercing the flexible implant 20 and gripping features to enhance the contact between the anchor plate 30 and bone anchor 50. In the embodiment shown in FIG. 6A, the penetrating members 36 comprise a series of teeth or serrations integrally formed on the bottom surface 34 of the anchor plate 30 to pierce the top surface of the flexible implant 20. The teeth or serrations 36 may be slightly rounded in some embodiments to prevent damaging threads or fibers, yet sufficiently pointed to penetrate the top surface 22 of the bone plate 20. The top surface 32 of the anchor plate 30 is smooth or textured to improve gripping contact between the bone anchor 50 and the anchor plate 30.

In the embodiment shown in FIG. 6B, the penetrating members 36 comprise one or more bone spikes that extend from the bottom surface 34 of the anchor plate 50 to pierce the top surface of the flexible implant 20. The length of the bone spikes allows the bone spikes to penetrate all the way through the anchor plate 30 and into the vertebral member 102. The top surface 32 of the anchor plate 30 is smooth or textured to improve gripping contact between the bone anchor 50 and the anchor plate 30.

In the embodiment shown in FIG. 6C, the penetrating members 36 comprise a series of teeth or serrations integrally formed on the bottom surface 34 of the anchor plate 50 to pierce the top surface of the flexible implant 20. The top surface 32 of the anchor plate 30 includes a series of serrations or teeth 38 to engage corresponding teeth or serrations on the bone anchor 50. When the bone anchor 50 is tightened against the anchor plate 30, the teeth or serrations 38 on the top surface 32 of the anchor plate 30 engage corresponding teeth on the bone anchor 50 to prevent the bone anchor 50 from backing off.

The anchor plate 30 illustrated in FIG. 6D includes one or more bone spikes on its bottom surface 34 functioning as penetrating members 36. The bone spikes are designed to penetrate all the way through the bone plate 20 and into the vertebral member as previously described. The top surface 32 of the anchor plate 30 includes a series of serrations or teeth 38 to engage corresponding teeth or serrations on the bone anchor 50. When the bone anchor 50 is tightened against the anchor plate 30 the teeth or serrations engage and prevent the bone anchor 50 from backing off.

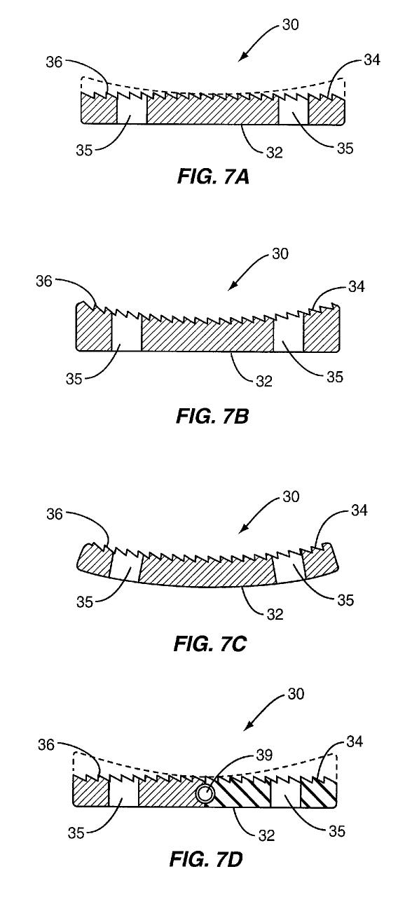

FIGS. 7A-7D are longitudinal cross-sections of exemplary anchor plates 30 taken through line VII-VII of FIG. 2. The anchor plate 30 shown in FIG. 7A is flat in cross-section. The anchor plate 30 in this embodiment is made of a material that inelastically deforms when the bone anchors 50 are tightened down to conform to the contour of the vertebral member 102. Suitable materials for a deformable anchor plate 30 includes polymers and metals, such as polyetheretherketone (PEEK), polyetherketoneketone (PEKK), other polyaryletherketone (PAEK) materials, carbon-reinforced PEEK, polyetherimide, polyimide, polyethylene terephthalate (PET), polyester, polylactic acid materials (PLA and PLDLA), polysulfone, ultra-high molecular weight polyethylene (UHMWPE), polyurethane, silicone, silicone-polyurethane, cobalt-chromium alloys, stainless steel alloys, titanium, titanium alloys, nickel titanium alloys, NITINOL, or their combinations. In some embodiments, certain materials, such as metals, may be deformable when their thickness is reduced below a certain amount.

In the embodiment shown in FIG. 7B, the bottom surface 32 of the anchor plate 30 is contoured to conform to the general shape of a vertebral member 102, while the top surface is flat in cross-section. Anchor plates 30 may be provided with different sizes and different curvatures so that the surgeon can select an anchor plate 30 of appropriate dimension and shape during a surgical procedure. In this embodiment, the anchor plate 30 could be deformable or non-deformable.

FIG. 7C illustrates an anchor plate 30 that is curved in cross-section. As with the previous embodiment, anchor plates 30 of different dimension and curvatures may be provided so that the surgeon can select an appropriate anchor plate 30 during a surgical procedure. The anchor plate 30 in this embodiment may be deformable or non-deformable.

FIG. 7D illustrates an embodiment of the anchor plate 30 where the right and left sections of the anchor plate 30 are pivotably connected. In this embodiment, a hinge pin 39 pivotably connects the right and left sections of the anchor plate 30. The hinge pin 39 allows the right and left sections to pivot relative to one another to conform to the vertebral member 102 when the bone anchors 50 are tightened. In this embodiment, the anchor plate 30 may be made of a deformable or non-deformable material. In the embodiment of FIG. 7D, the right and left sections are constructed of different materials. In another embodiment, the sections are constructed of the same materials.

Exemplary materials for the anchor plate 30 include polymers, such as polyetheretherketone (PEEK), polyethylene terephthalate (PET), polyester, polyetherketoneketone (PEKK), polylactic acid materials (PLA and PLDLA), polyaryletherketone (PAEK), carbon-reinforced PEEK, polysulfone, polyetherimide, polyimide, ultra-high molecular weight polyethylene (UHMWPE), and/or cross-linked UHMWPE, and combinations thereof. Metals or ceramics can also be used, such as cobalt-chromium alloys, titanium, titanium alloys, nickel titanium alloys, NITINOL, memory wire and/or stainless steel alloys, calcium phosphate, alumina, and/or pyrolytic carbon, and combinations thereof.

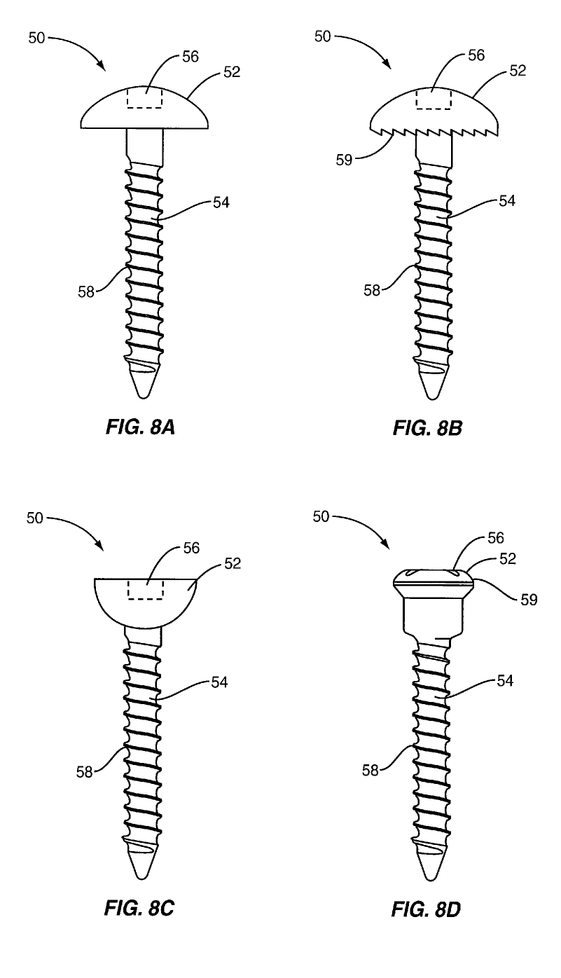

FIGS. 8A-8D illustrate exemplary bone anchors 50. In FIG. 8A, the bone anchor 50 comprises a bone screw including a head 52 and a shank 54. The head 52 is provided with a slot or socket 56 for a torque-applying instrument. The shank 52 includes threads 58 for screwing the bone anchor 50 into the vertebral member 102. In this embodiment, the bottom surface of the head 52 is flat and adapted for use with anchor plates 30 including a generally flat top surface 32. Those skilled in the art will appreciate, however, that the bottom surface of the head 52 and/or the top surface 32 of the anchor plate 30 may be roughened to enhance contact between the head 52 and the top surface 32 of the anchor plate 30. Alternately, lock washers or similar locking devices can be used to prevent the bone anchor 50 from backing off after insertion of the stabilizing system.

FIG. 8B illustrates another embodiment of the bone anchor 50. This embodiment comprises a bone screw including a head 52 and shank 54. The head 52 includes a slot or socket 56 for a torque-applying instrument. The shank 54 includes threads 58 for penetrating and screwing into the vertebral member 102. In this embodiment, the bottom surface of the head is provided with a series of teeth or serrations 59 which are configured to engage corresponding teeth or serrations 38 on the top surface 32 of the anchor plate 30. Thus, the teeth or serrations 38, 59 cooperate to prevent the bone anchor 50 from backing off after insertion.

FIG. 8C illustrates an embodiment with the head 52 being hemispherical with a curved bottom surface. In one embodiment, the curved bottom surface matches a corresponding curved opening within the top surface of the anchor plate 30. The head 52 includes a flat surface that may be substantially flush with top surface of the anchor plate 30.

FIG. 8D illustrates an embodiment with the head 52 including a lip 59 that is contacted by an anti-backout mechanism as will be explained in detail below.

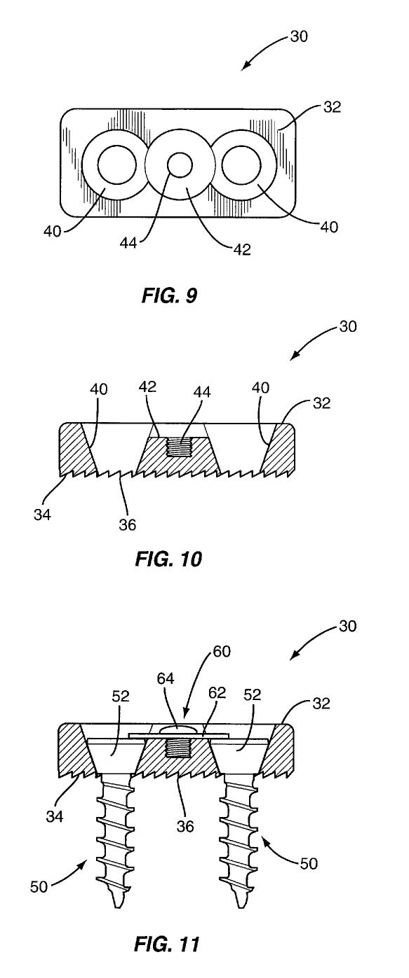

FIGS. 9-11 illustrate another exemplary embodiment of an anchor plate 30. In this embodiment, the anchor plate 30 has a flat top surface 32 and a serrated bottom surface 34 including a plurality of serrations or teeth that function as penetrating members 36. The anchor plate 30 includes conical openings 40 configured to receive bone screws 50 including conical heads 52. A recessed seat 42 is formed between the conical openings 40. A threaded opening 44 is formed in the recessed seat 42 to receive a locking screw 64. The locking screw 64 along with locking washer 62 comprises a locking assembly 60. After insertion of the bone anchors 50, the locking washer 62 is attached to the recessed seat 42 by locking screw 64. The locking washer 62 overlaps the heads 52 of the bone anchors 50 to prevent the bone anchors 50 from backing out.

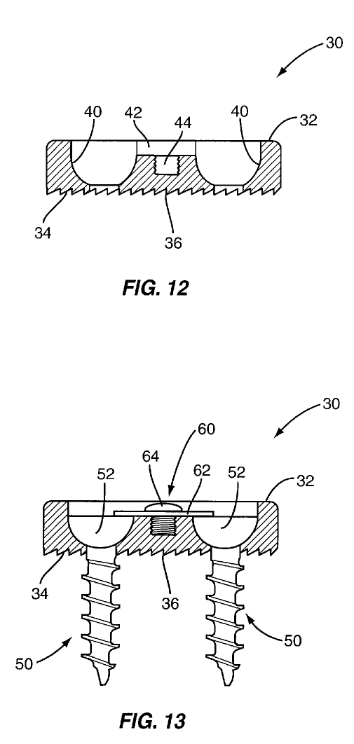

FIGS. 12-13 illustrate another embodiment of an anchor plate 30 that includes hemispherical openings 40 configured to receive bone screws 50 including hemispherical heads 52. The shape of the screw heads 52 corresponds with the openings 40 such that the top of the heads 52 is flush with or recessed within the top surface 32 of the anchor plate 30. The locking washer 62 overlaps the heads 52 of the bone anchors 50 to prevent the bone anchors 50 from backing out.

In an alternate embodiment, the locking washer 62 may be attached to the recessed seat 42 before insertion of the bone anchors 50. In this embodiment, the outer edge of the locking washer 62 is designed to flex downwardly and/or inwardly when the bone anchor 50 is inserted and to return to its original condition when the bone anchor 50 has passed. The locking washer 62 is designed so that it will not flex outwardly. Thus, the bone anchors cannot back out after they are inserted.

Another embodiment of a locking structure to prevent backout of the bone anchors 50 includes adhesive bonding. The adhesive may be applied to the bone anchors 50 and/or anchor plate 30 to prevent the backout. In a similar embodiment, the anchor plate 30 may be deformed using heat, force, or solvents to inhibit withdrawal of the anchors 50. Examples of adhesive bonding are disclosed in U.S. Pat. Nos. 6,605,090 and 7,172,593 each incorporated herein by reference in their entireties.

Various types of fasteners may be used to attach the implants 20 and plates 30 to the vertebral members 102. Embodiments described above include bone anchors 50 in the form of screws that include a head and shaft portion. Other types of fasteners may include but are not limited to pins, rivets, staples, tethers, deployable anchor, and wires.

Various embodiments have been described for the purpose of understanding the principles of the application. The embodiments are intended to be illustrative only and alternate embodiments applying the principles of the present application will be readily apparent to those skilled in the art. Therefore, the descriptions of specific embodiments herein are not intended to imply any limitations on the application not explicitly stated in the claims.

What is claimed is:

1. A stabilizing system for stabilizing a patient's spinal column, said stabilizing system comprising:
a flexible implant configured for attachment to a vertebral member, said flexible implant including a bottom surface and a top surface;
an anchor plate for attaching the flexible implant to the vertebral member, said anchor plate including a bottom surface with one or more projections configured to penetrate the top surface of the flexible implant; and
at least one fastener to secure the anchor plate to the vertebral member.

2. The stabilizing system of claim 1 wherein said one or more projections on said bottom surface of said anchor plate comprise one or more teeth.

3. The stabilizing system of claim 1 wherein said one or more projections on said bottom surface of said anchor plate comprise one or more serrations.

4. The stabilizing system of claim 1 wherein said one or more projections on said bottom surface of said anchor plate comprise one or more spikes.

5. The stabilizing system of claim 1 wherein said projections are configured to penetrate through said anchor plate and into the vertebral member.

6. The stabilizing system of claim 1 wherein said anchor plate is deformable.

7. The stabilizing system of claim 1 wherein said anchor plate is non-deformable.

8. The stabilizing system of claim 7 wherein said anchor plate includes a contoured bottom surface shaped to conform to a vertebral member.

9. The stabilizing system of claim 7 wherein the cross-sectional shape of the anchor plate conforms to the shape of a vertebral member.

10. The stabilizing system of claim 1 wherein said anchor plate includes a hinge pivotally connecting first and second sections of said anchor plate.

11. The stabilizing system of claim 1 wherein the one or more fasteners comprise one or more bone screws and wherein the anchor plate includes one or more openings through which the bone screws pass.

12. The stabilizing system of claim 11 wherein the bone screws include a head configured to contact a top surface of the anchor plate.

13. The stabilizing system of claim 12 wherein the top surface of the anchor plate includes a gripping feature to enhance gripping contact between the head of the bone screw and the top surface of the anchor plate.

14. The stabilizing system of claim 13 wherein the gripping feature comprises one or more teeth.

15. The stabilizing system of claim 13 wherein the gripping feature comprises a textured surface on the anchor plate.

16. The stabilizing system of claim 13 wherein the head of the bone screw includes a gripping feature to enhance gripping contact between the head of the bone screw and the top surface of the anchor plate.

17. The stabilizing system of claim 11 further comprising at least one locking device for locking said bone screws in place following insertion of said bone screws.

18. The stabilizing system of claim 17 wherein said locking device comprises a locking washer overlapping said bone screws and a locking screw to secure the locking washer to the anchor plate.

19. A stabilizing system for stabilizing a patient's spinal column, said stabilizing system comprising:
an implant configured to attach to a plurality of vertebral members, the implant including a bottom surface that contacts the plurality of vertebral members and a top surface;
first and second holes each extending through the implant;
an anchor plate positioned against the top surface of the implant and including projections that penetrate into the implant, the anchor plate further including a plurality of holes that align with the first and second holes in the implant; and
first and second fasteners that extend through each of the first and second hole in the implant and the plurality of holes in the anchor plate to secure the implant to one of the plurality of vertebral members;
the anchor plate extending around each of the plurality of holes to distribute the forces on the implant applied by the plurality of fasteners.

20. The stabilizing system of claim 19 wherein the anchor plate is substantially rectangular.

21. The stabilizing system of claim 19 wherein the anchor plate is substantially hourglass shaped.

22. The stabilizing system of claim 19 further comprising a second anchor plate positioned on the top surface of the implant to distribute forces applied by a third fastener that secures the implant to the second of the plurality of vertebral members.

23. A stabilizing system for stabilizing a patient's spinal column, said stabilizing system comprising:
an implant configured to extend between first and second vertebral members, the implant including a bottom surface that contacts the vertebral members and a top surface;
first and second anchor plates positioned against the top surface of the implant and including projections that penetrate into the implant; and
a first fastener that extends through the first anchor plate and the implant to secure the implant to one of the first vertebral member;
a second fastener that extends through the second anchor plate and the implant to secure the implant to one of the second vertebral member;
the first and second anchor plates positioned to distribute the forces on the implant applied by the first and second fasteners.

24. The stabilizing system of claim 23 wherein the implant is sized to extend across an intervertebral disc positioned between the first and second vertebral members.

25. The stabilizing system of claim 23 wherein each of the first and second anchor plates include a hole that align with the first and second holes in the implant.

26. A method for attaching a flexible implant to a vertebral member, said method comprising:
disposing the flexible implant between the vertebral member and an anchor plate, said anchor plate being configured to distribute a clamping force generated by a force applying member over a top surface of said flexible implant and including a bottom surface with one or more projections configured to penetrate the top surface of the flexible implant; and
applying said clamping force to said anchor plate to secure the flexible implant between the anchor plate and the vertebral member and to force said one or more projections on said bottom surface of said anchor plate into a top surface of the flexible implant.

27. The method of claim 26 wherein said one or more projections penetrate through said anchor plate and into the vertebral member.

28. The method of claim 26 wherein said one or more projections comprise one or more teeth.

29. The method of claim 26 wherein said one or more projections comprise one or more serrations.

30. The method of claim 26 wherein said one or more projections comprise one or more spikes.

31. The method of claim 26 further comprising deforming said anchor plate to conform to said vertebral member when said clamping force is applied.

32. The method of claim 26 further comprising pivoting first and second sections of said anchor plate about a hinge to conform said anchor plate to said vertebral member when said clamping force is applied.

33. The method of claim 26 wherein applying said clamping force to said anchor plate to secure the flexible implant between the anchor plate and the vertebral member comprises urging a contoured surface of said anchor plate shaped to conform to a vertebral member into contact with said flexible implant.

34. The method of claim 26 wherein applying said clamping force to said anchor plate to secure the flexible implant between the anchor plate and the vertebral member comprises inserting one or more fasteners through corresponding openings in said anchor plate and tightening said fasteners against said anchor plate.

35. The method of claim 34 further comprising engaging a gripping feature on a top surface of said anchor plate with a head of said one or more fasteners.

36. The method of claim 35 wherein engaging a gripping feature on a top surface of said anchor plate with a head of said one or more fasteners comprises engaging a gripping feature on a top surface of said anchor plate with a gripping feature on said head of said one or more fasteners.

37. The method of claim 34 further comprising locking said fasteners in place with a locking device following insertion of said fasteners.

38. The method of claim 37 wherein locking said fasteners in place with a locking device following insertion of said fasteners comprises disposing a locking washer above said fasteners.
